(19) United States
(12) Patent Application Publication
(10) Pub. No.: US 2006/0038978 A1
(43) Pub. Date: Feb. 23, 2006

(54) Automatic candling machine

(76) Inventors: David Zweig, Randolph, NJ (US); Kenneth C. Christiansen, Rockaway, NJ (US)

Correspondence Address: CARELLA, BYRNE, BAIN, GILFILLAN, CECCHI,;STEWART & OLSTEIN, 5 BECKER FARM ROAD, ROSELAND, NJ, 07068, US

(21) Appl. No.: 11/205,566

(22) Filed: Aug. 17, 2005

Related U.S. Application Data
(60) Provisional application No. 60/602,473, filed on Aug. 18, 2004.

(51) Int. Cl. G01N 33/08; A01K 43/00

(52) U.S. Cl. 356/56

(57) ABSTRACT

Eggs are loaded on a tray in rows and columns and conveyed to a candling station having a plurality of candling locations, each location for candling a portion of eggs on a tray, a different tray at each location. Different rows of eggs of a tray are candled in the different locations so that all eggs of each tray are candled by all of the locations. Each location has a plurality of digital video cameras, each camera focused on a portion of eggs in a row, the eggs being imaged being located in one or more rows. The eggs of each row being imaged are lifted by an associate lifting assembly which selectively rotates the egg for imaging in two opposite imaging orientations by each camera. A controller operates the cameras, lifting and rotating assemblies, positions lights adjacent to each egg during imaging, and evaluates the quality of each egg by comparing veins, air sac location and embryo of each imaged egg to a reference to produce a quality value for each egg and from this value assessing whether the egg is acceptable or unacceptable. The unacceptable eggs are removed from each tray by a suction head operated by the controller.

This application claims the benefit of provisional application Ser. No. 60/602,473 filed Aug. 18, 2004, entitled “Automatic Candling Machine” incorporated by reference herein in it's entirety.

### FIELD OF THE INVENTION

This invention relates to candling apparatuses, i.e., apparatus for egg inspection.

### BACKGROUND OF THE INVENTION

It has long been known in the poultry field that the viability or acceptability of an egg can be determined through the use of a process known as candling. Candling utilizes light to illuminate the interior of an egg, e.g. to determine whether or not the egg is viable in the vaccine industry or alternatively not viable in the consumption industry.

Traditionally, candling has been accomplished manually. This involves an operator causing a light to come in contact with the shell of an egg to illuminate the egg and, while the interior of the egg is illuminated by the light, inspecting the egg to determine if there is blood flow or growth, the presence or absence of an air sac, or other conditions which would make the egg acceptable for the purpose intended or unacceptable for the purpose intended.

Many attempts have been made over the years in this field to have candling done automatically as distinguished from being done manually. None has been a success.

Candling is used in different applications depending upon the particular function and inspection that is desired to be achieved. For example, in the pharmaceutical industry candling is used to identify viable eggs that are suitable for use in the manufacture of vaccines. By way of comparison, candling is used in the edible egg industry to determine the presence or absence of a viable embryo so as to separate eggs not suitable for human consumption from eggs that are. Other applications of this technology will become obvious to those having skill in these arts based upon the disclosure of the invention as set out below.

Accordingly, it is an object of the present invention to provide a method and apparatus for automatically inspecting eggs through the use of light sources and automatic light sensing apparatus.

It is a further object of the present invention to provide such an apparatus that is controlled automatically through the use of novel software.

These objects and others that will become clear are achieved with the method and apparatus of the present invention that may include a conveyor for advancing trays of eggs through a candling machine, at least one candling station for illuminating the interior of eggs being candled and imaging, e.g. by digital camera, the eggs from one or more angular perspectives, identifying eggs with defects through the use of automatic apparatus, and removing defective eggs for disposal.

An egg candling apparatus according to an aspect of the present invention comprises an automatic imaging system for producing an image of each egg of a plurality of eggs manifesting the quality of each egg and an egg quality evaluation system coupled to the imaging system for automatically determining which of the imaged plurality of eggs have at least a given quality value.

In one aspect, an egg separating system is coupled to the imaging system for separating those eggs that have at least the given quality value from those that do not have that value.

In a further aspect, the imaging system includes a plurality of digital video cameras each of which is selectively focused on one or more of the eggs.

In a further aspect, the imaging system includes a plurality of egg positioning assemblies for lifting and rotating each egg, a plurality of egg illuminating assemblies to illuminate each lifted egg and the plurality of the digital video cameras to image the illuminated egg interiors.

In a still further aspect, each egg is first imaged in a first relative angular orientation to the cameras and then rotated to a second angular orientation for imaging by the cameras.

In a further aspect, an egg holding tray is included for holding the eggs in rows and columns, each tray having an opening therein for receiving therethrough a corresponding egg lifting and rotating assembly.

In a further aspect, included are a light assembly for illuminating the eggs, egg positioning assemblies for abutting each egg against a corresponding light assembly, and a drive motor for selectively rotating and lifting the eggs for imaging by the cameras.

In a further aspect, the eggs are positioned in a plurality of columns and rows on a tray, the positioning assemblies, lights and cameras being arranged to selectively image the eggs in at least one row of eggs on a tray.

A plurality of locations are preferably included wherein all of the eggs on a tray are imaged in successive ones of the locations, each location for imaging at least one different row of eggs of the plurality of rows.

Preferably, each camera is arranged to image a plurality of eggs in a row.

Preferably, in another aspect, the imaging system is arranged to image the veins, air sac and location of the embryo of each egg and includes a controller to compare the veins, air sac and embryo to a reference.

In a further aspect, included are a conveyor; at least one tray holding a plurality of eggs in rows and columns for displacement by the conveyer; at least one candling station for candling the plurality of eggs, the at least one station including the imaging system, the imaging system including a digital video camera for imaging at least a portion of the plurality of eggs at a time, the imaging system further including egg lifting and rotating assemblies for lifting the portion of the eggs and an illuminating system for illuminating the interiors of the lifted portion of eggs for the camera imaging; and a controller for operating the imaging and illuminating systems and the quality evaluation system.

In a still further aspect, included is a controller for operating the imaging and evaluation systems.

Preferably, included in the controller are a CPU and a data base containing reference data identifying the features of a viable egg and programmed instructions for comparing the features of each of the illuminated eggs with reference data features.

A method for candling eggs comprises automatically producing an image manifesting the quality of each egg of a plurality of eggs and automatically evaluating the image of each egg to determine which eggs have at least a given quality value.

### BRIEF DESCRIPTION OF THE DRAWINGS

A clearer understanding of the present invention may be had from the following detailed description, particularly when read in light of the following drawings, wherein:

FIG. 1 is a plan view of an automatic candler according to an embodiment of the present invention shown with the top of the candler removed:

FIG. 2 is a front elevational view of the automatic candler of FIG. 1 with the front doors of the candler housing removed for visual access;

FIG. 3 is a side elevational view of the automatic candler of FIG. 1 showing a representative candling station with the side doors removed for visual access;

FIGS. 4 and 4a are side sectional elevation views of a single candling station respectively showing the egg illuminating lights prior and subsequent to lowering into engagement with the eggs according to an embodiment of the present invention;

FIG. 5 is a front sectional elevational view of the station of FIG. 4 showing the egg positioning portion of the apparatus of the present invention;

FIG. 6 is a photograph of an egg imaged by the imaging system of the present invention;

FIG. 7 is a photograph of four eggs imaged by the imaging system of the present invention similar to the photograph of FIG. 6 and showing unacceptable and acceptable egg images;

FIG. 8 is a schematic block diagram of the electronic control system of the present invention; and

FIG. 9 is a flow chart of the programming utilized in the control system operating the candler of FIGS. 1 and 2.

### DETAILED DESCRIPTION OF THE PREFERRED EMBODIMENTS

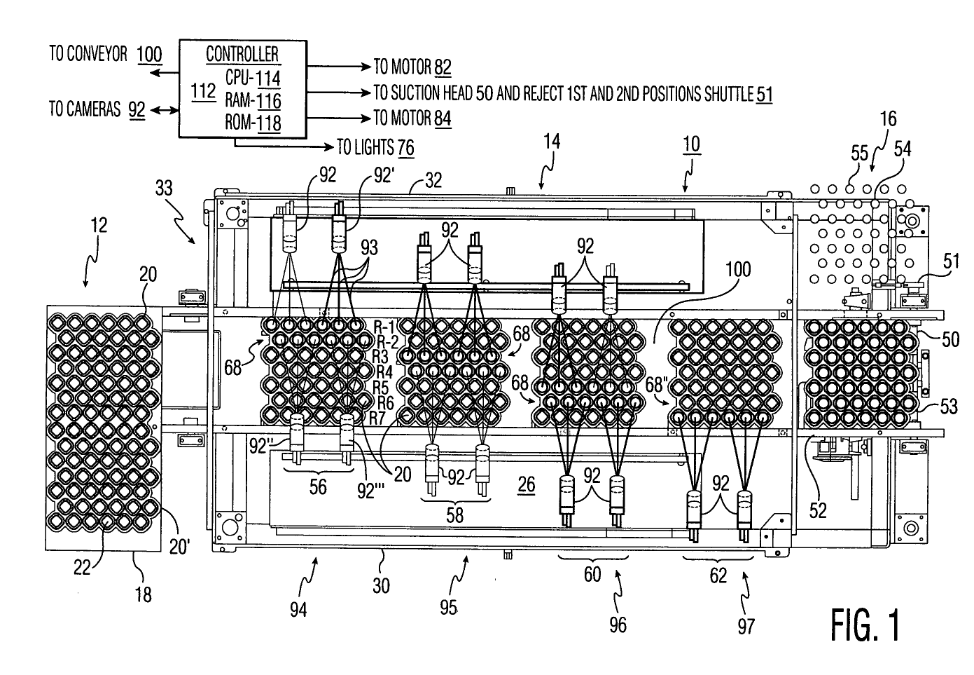

Referring now to the drawings, and in particular to FIG. 1, an automatic candler according to the present invention is shown and designated generally by the reference numeral 10.

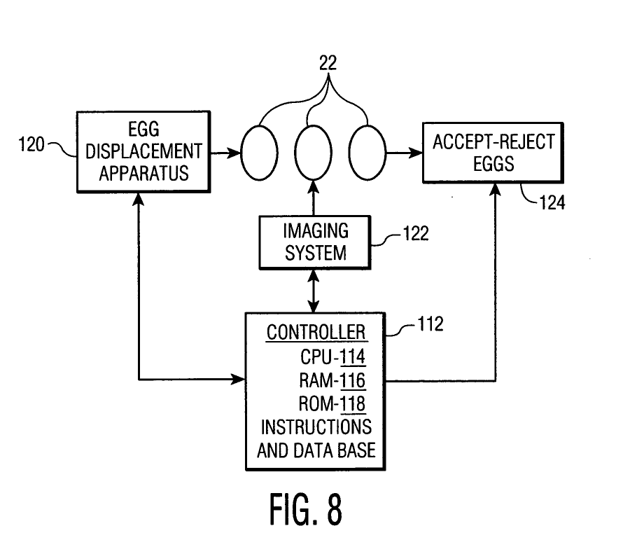

Candler 10 is operated automatically under control of controller 112. Controller 112 includes, FIG. 8, a CPU 114, RAM 116 and ROM 118. The CPU is a microcontroller and can be of any commercially available microcontroller design. The RAM and ROM have programmed instructions for operating the candler 10. Included in controller 112 is a data base representing good egg images which serve as references for the candler 10 in a manner to be explained.

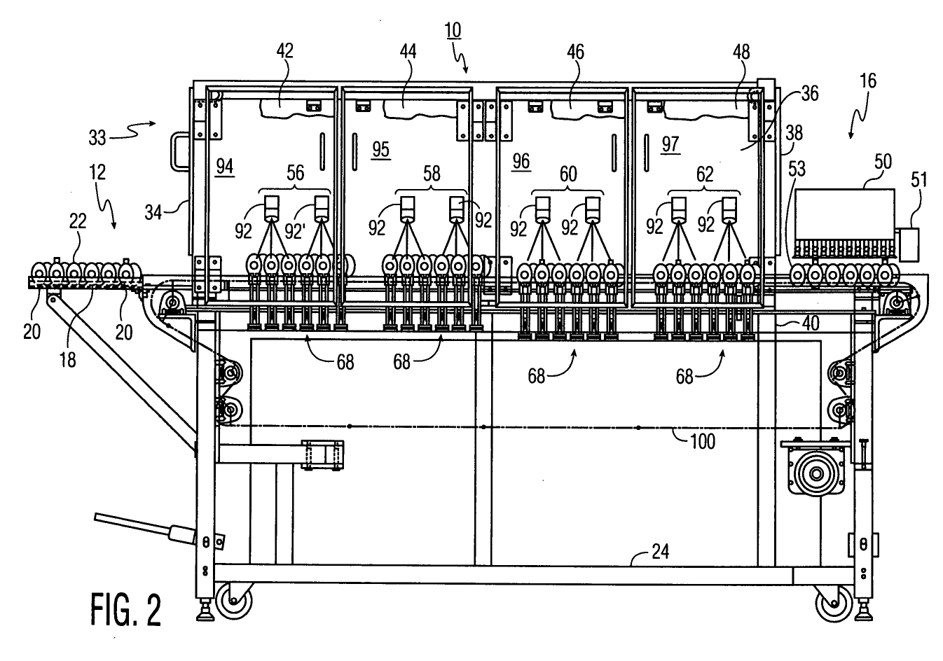

In FIGS. 1 and 2, candler 10 includes an operator station 12, a multi-position candler station 14, and an automatic reject shuttle station 16.

Operator station 12 includes a feed table 18 on which an operator places trays 20 of eggs 22 to be candled. The feed table 18, while shown as a slide table, may be a roller table, a conveyor, a slide table or any of the many others known generally to those skilled in these arts. In this regard, feed table 12 is conventional and does not form a part of the present invention.

The trays 20 as shown in the embodiment of FIGS. 1-3 are designed to hold forty-two eggs each with rows of six, i.e. the rows extending longitudinally (left to right) FIGS. 1 and 2, and columns of seven, i.e. columns extending transversely (top to bottom of the figure) as shown in FIG. 1, with the eggs in the adjacent columns staggered as shown. Trays carrying different numbers of eggs are contemplated by the invention and more or fewer eggs can be candled using the inventive apparatus to practice the inventive method.

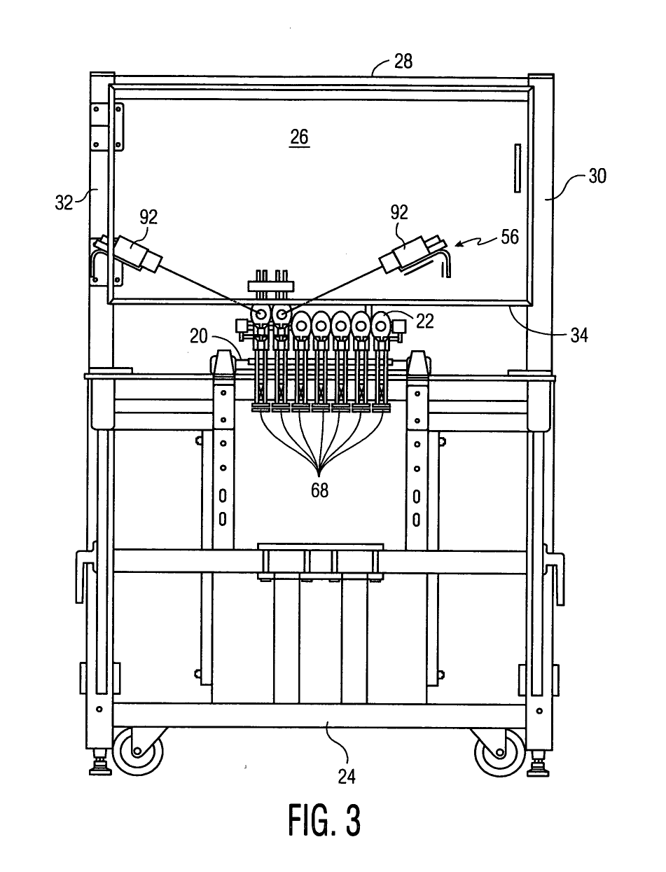

Multi-position candling station 14 is a generally box-shaped structure comprising a frame 24 which is enclosed to define an interior 26 for operably receiving the candling equipment. The station 14 Interior 26 is enclosed by a top casing 28 and side walls 30, 32, FIG. 3. Entry end 33 of the candler station 14 at the operator station 12 is partially closed by a door 34 which is sized to provide a closure defining an opening sufficiently large to permit passage of eggs 20 to be candled from the operator station 12 to the interior 26 of the candling station. Exit end 36 of the candling station 14, FIG. 2, also is partially closed by an exit door 38 to define an exit opening 40 such as to permit egress of candled eggs from the candling station interior 26.

Side wall 30 of the multi-position candler station 14, FIG. 2, is provided with four doors 42, 44, 46 and 48, each door located to correspond to a respective location 94, 95, 96, and 97 of a candling position.

Mounted on frame 24 just outside the exit door 38 is an automatic reject station 16. As is discussed below in detail, the candling procedure identifies eggs that are not suitable for the purpose intended, e.g. for use in vaccine, or for human ingestion. The locations of such eggs are communicated to a suction head 50, FIG. 2, by controller 112, FIG. 1, which head is slidably mounted to move from a first position 52 over a tray 53 of eggs which has exited through exit opening 40 to a second position 54 over a rejected egg receiving container 55. Eggs identified for disposal by controller 112 are picked up at the first position 52 by the egg suction head 50 under control of controller 112 and carried to the second position 54 where they are dropped into a container 55 under control of controller 112 and thereafter discarded. The remaining good eggs are then forwarded for packaging.

Operably located within interior 26 of candle station 14 are four candling heads 56, 58, 60 and 62 each at a respective candling location 94-97. Each candling head comprises a plurality of cameras 92 and egg positioning assemblies 68 as described in detail below. Each of candling heads 56, 58 and 60 candles two rows of eggs while candling head 62 candles a single row.

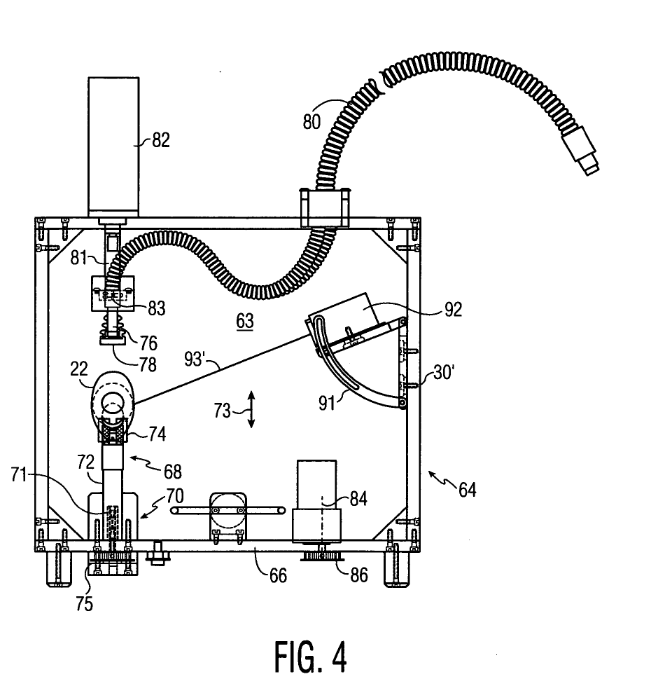

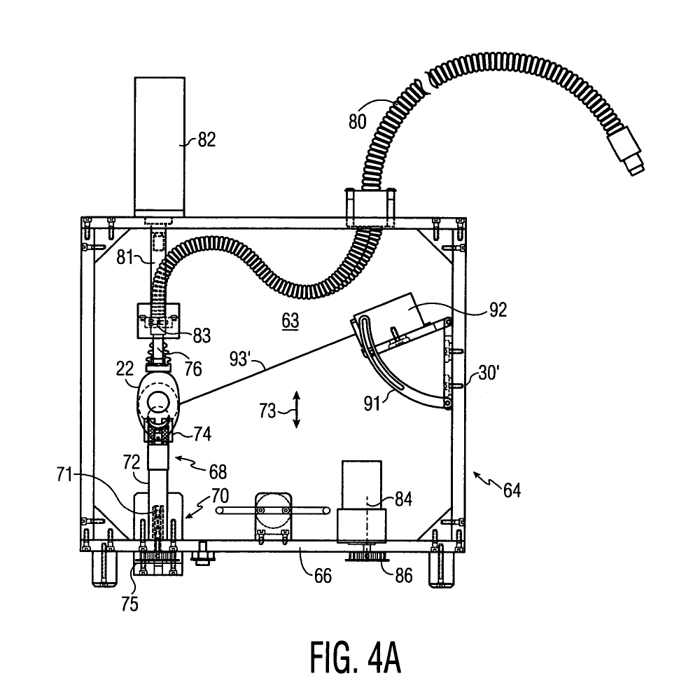

In FIGS. 4 and 5, there is shown, for purposes of exemplary structural and operational disclosure, a single row candling assembly 64. Each assembly 64 includes four egg positioning assemblies 68 for positioning four eggs simultaneously and a camera 92 responsive to a beam 93′ of light from illuminated eggs 22. The beam 93′ contains the image of the interiors of eggs 22. The camera is focused on all four of the eggs 22 which are positioned and held by assemblies 68 of this embodiment. All of the locations 94-97 of FIG. 1 employ a candle assembly generally similar to assembly 64, but which differ as follows.

Each of the cameras 92 of FIG. 1 in the four locations 94-97 focuses simultaneously on three eggs, not four, as does camera 92 of assembly 64, FIG. 4. A further difference is that the assembly 64, FIGS. 4 and 5, has four egg positioning assemblies 68 that position four eggs simultaneously in a single row. In FIG. 4, the candling assembly 64 has one camera 92. In contrast, the locations 94-96 each have four cameras 92 at each head 56, 58 and 60, with two cameras on opposite sides of the trays 20. Each location 94-96 has twelve egg positioning assemblies 68 which simultaneously position twelve eggs in two adjacent rows of six eggs each. A further difference is that the egg positioning assemblies 68 of location 97 position simultaneously one row of six eggs and the head 62 has two cameras 92 as compared to one in assembly 64. Among all of the assemblies 68 in candler 10, all eggs are thus positioned for processing by the assemblies 68 associated with each of the corresponding eggs 22. The egg positioning assemblies 68 are all substantially the same.

The operation of the candling assemblies of FIGS. 1-3 and 4-5 is substantially identical notwithstanding the different number of eggs being processed. Specifically, the operation includes the steps of positioning eggs 22 in a tray and introducing the tray with eggs into the candling apparatus such that the eggs are in vertical alignment with the egg positioning assemblies 68. With the eggs so aligned, the telescoping tubes 72 are actuated, e.g., hydraulically, pneumatically, or mechanically, to raise the eggs until their upper shell surfaces engage the surfaces of the corresponding cups 78 of light sources 76. The light sources 76 are then actuated and cameras 92 are actuated to commence the inspection process as is discussed below in detail. Upon completion of the inspection of the eggs in one orientation relative to the cameras, the eggs are lowered such as to clear the cups 78 and rotated 180°. With the eggs in this rotated position, the inspection process is repeated.

Upon completion of the inspection procedure, the telescoping tubes 72 are actuated to lower the eggs back into the trays 20. The trays 20 are then advanced within the candling apparatus where the process is repeated with respect to the second pair of rows of eggs. This process continues until all of the eggs have been inspected and unacceptable eggs identified. The unacceptable eggs are withdrawn from the tray and discarded.

In FIG. 1, each location 94-96 has an egg positioning assembly 68 that positions two rows of eggs simultaneously. Location 97 is similar to assembly 64 in that its egg positioning assembly 68 positions the eggs in only one row, notwithstanding there are six eggs that are positioned in location 97, FIG. 1, as compared to the four eggs positioned by assemblies 68 of FIG. 4. All heads including the candle positioning assemblies are otherwise substantially identical at locations 94-96. Thus, each of locations 94-96 has four cameras and egg positioning assemblies 68 that position twelve eggs in two rows. Location 97 has two cameras and an egg positioning assembly 68 that positions six eggs.

Candle assembly 64, FIGS. 4 and 5, includes a base 66 on which is mounted four egg positioning assemblies 68 each comprising an egg rotating and lifting assembly 70, FIG. 5. Each egg rotating and lifting assembly 70 is identical in all embodiments. A representative assembly 70, FIG. 5, comprises a telescoping tube 72, tube 72 having a rubber cap 74 mounted on its upper end. Rubber cap 74 is sized to receive an egg 22 and retain the egg during vertical displacement and also during rotation of the corresponding egg as discussed below.

Each tray 20 (not shown in FIGS. 4 and 5) has an opening (not shown) in which an egg 22 is positioned. The tubes 72 are aligned with respect to the trays 20 so that the openings in a tray are aligned with a corresponding different one of the four corresponding telescoping tubes 72. Each of the telescoping tubes 72 and caps 74 are dimensioned to pass through the aligned openings in the corresponding trays 20. The tubes 72 lift the associated eggs 22 from their positions on the tray 20 to a position above the tray. The light sources 76 in the meantime have been lowered by motor 82 to the position shown in FIG. 4a. The lifted eggs at this time engage the resilient lowered light source caps 78, FIG. 4a. In FIGS. 4, 4a and 5, the eggs 22 shown in phantom are in their lowermost position whereas the eggs in solid line are shown lifted by the corresponding tube 72. The small circle in each egg represents the yoke. Each telescoping tube 72 of assembly 70 has a lifting mechanism 71 associated therewith. Mechanism 71 may be any known device such as a mechanical rotating screw arrangement, a hydraulic actuated device or other device for telescoping and lifting the cap 74 in response to activation of the lifting device. In addition to being translated vertically, the eggs 22 can be rotated through the actuation of mechanism 71. The mechanism 71 is driven by a motor 84 and a toothed drive belt (not shown) via toothed drive pulley 86 attached to motor 84 and toothed driven pulleys 75 attached to each mechanism 71. Each assembly 68 thus includes assembly 70 and a toothed driven pulley 75. Obviously the drive belt will have different lengths according to the number of pulleys being driven in the different embodiments. All pulleys are driven in a predetermined direction and for a given angular rotation by controller 112, FIG. 1, to insure each tube 72 is lifted to its desired elevation and also to rotate the tubes 72 180° after inspection of one side of the eggs is complete. The tubes are lowered by a reverse rotation of the corresponding tubes 72. All of the motor 84 action is under control of controller 112, FIG. 1.

Coaxially aligned above each telescoping tube 72 is a light source 76 disposed in a cup-shaped element 78 and supplied with electrical power through a flexible cable 80. In FIG. 5, each of the four light sources 76 associated with each tube 72 is mounted on a vertically movable horizontal bracket 77. The horizontal bracket 77 is mounted at its opposite ends on vertically oriented guide rods 79. The bracket 77 slides vertically on guide rods 79 for reciprocation in directions 73. A motor 82 or other suitable displacement device is coupled to a drive shaft 81 via a vertical drive mechanism 83. The mechanism 83 may be a male and female screw arrangement or a gear mechanism (not shown) or the mechanism may be pneumatically or hydraulically operated (not shown) such as to displace the shaft 81 vertically in response to operation of the mechanism 83.

In the embodiment of FIGS. 4 and 5, the shaft 81 is coupled to bracket 77 via mechanism 83 for displacing the bracket 77 in directions 73 under control of controller 112. Displacement of the bracket 77 moves the light source 76 in directions 73 to adjust the position of the light source 76 relative to the cap 74 and received egg 22 to illuminate the associated egg 22 interior as known in this art. The eggs abut the light source cap 78. The light source 76 to egg 22 spacing is predetermined as known for a given illumination magnitude. This displacement of the light sources against the eggs positions and holds the light sources 76 at the desired spaced position from the eggs 22 being examined. The candlepower of the light sources, spacing of the sources to the eggs and size of the eggs relationships are known in this art. Motor 82 may be an air motor, an electric motor or other such device suitable to displace the frame 77 and the sources 76 in response to rotation of the motor 82 shaft 81 under control of controller 112.

Each egg positioning assembly 68 rotates each corresponding lifted egg 22 at that station under direction of the controller 112 so that the egg surface may be viewed from different opposite angular perspectives from a given direction. Such rotation is achieved by motor 84 or other drive arrangement. In this embodiment of the invention, there is mounted on the shaft of the motor 84 a toothed drive pulley 86 which operably receives the toothed belt (not shown). Rigidly secured to the bottom of each telescoping tube 72 is the second toothed driven pulley 75. The belt is operably secured around the pulleys 75 and 86 such that when motor 84 operates under control of controller 112, it causes each telescoping tube 72 to lift and to rotate therewith the egg 22 in the selected row containing assemblies 68. All assemblies 68 are operated simultaneously by controller 112.

Mounted on the wall 30′ of assembly 64 via bracket assembly 91 is a camera 92 which is aligned to image digitally the four focused eggs 22 via beam 93′ from the eggs when the eggs are in position. A digital camera for this purpose has been found to be acceptable and its output is channeled to the controller 112, FIG. 1, as is discussed below. A plurality of cameras 92 are utilized to enable candling of a plurality of eggs simultaneously.

Thus, referring to FIG. 1, an automatic candler 10 is shown configured to candle consecutive trays carrying forty-two eggs each, i.e. seven rows, R1-R7, of six eggs each. Two trays 20, 20′ are shown at the operator station 12 awaiting insertion into the interior space 26, which can be done manually or automatically by apparatus not shown, or a combination of both methods. FIG. 1 also shows four trays of eggs, each tray located at one of four candling locations 94, 95, 96 and 97. Each candling location 94, 95 and 96 has twelve egg positioning assemblies 68, twelve light sources (see generally FIGS. 4 and 5) and four cameras 92. The twelve egg positioning tubes 72 of assemblies 68 are aligned with two rows of eggs associated with a corresponding tray. The fourth candling location 97 has six egg positioning tubes of assemblies 68, six light sources 76 and two cameras 92. The six egg positioning tubes 72 of assemblies 68 are aligned with one row of eggs associated with a corresponding tray.

The egg positioning assemblies 68 each comprising twelve egg positioning tubes 72 at each location 94-96 are associated with different ones of the rows of eggs. That is, each assembly 68 has a lifting and rotating tube 72. The assemblies 68 are associated with rows 1 and 2 at location 94, a second set of assemblies 68 have lifting and rotating tubes 72 associated with rows 3 and 4 at location 95, and so on. Therefore, among all of the assemblies 68 in candler 10 in locations 94-97, all rows and all eggs have a corresponding egg positioning assembly 68 and associated tubes 72 associated therewith. The egg positioning tubes 72 of all of the assemblies 68, FIG. 1, eventually become aligned with all of the eggs of a given tray when that tray is displaced to each of the locations 94-97 in sequence to complete the candling process.

Each of cameras 92, FIG. 1, is focused on three eggs which are in three next adjacent positions in a row. The focusing of the cameras is represented by image beams 93 to cameras 92. Beams 93 represent focused images from three eggs to the right in row 1 location 94. Thus, in candling location 94, an egg position assembly 68 is located to correspond to each egg of rows R-1 and R-2, row R-1 being most rearward and row R-2 being next most rearward (row R-7 being most forward). Each of the four cameras 92, 92′, 92″ and 92′″ at location 94 for example focuses on a set of three adjacent eggs, i.e. camera 92 focuses on the rearward most three eggs on the left side of row R-1, camera 92′ focuses on the rearward most three eggs on the right side of row R-1, camera 92″ focuses on the three eggs on the left side of more forward row R-2 and camera 92′″ focuses on the three eggs on the right side of more forward row R-1.

Thus, the four cameras of location 94 image twelve eggs from one perspective. These twelve images are stored by controller 112. The eggs are then each lowered and then simultaneously rotated 180° by controller 112 via the assemblies 68 and then raised by the assemblies 68. The same twelve eggs in rows R-1 and R-2 are then imaged from the opposite perspective by these same cameras 92, 92′, 92″ and 92′″.

The egg positioning assemblies 68 and cameras 92 are set up in candling locations 95 and 96 to operate in the same manner as set out with regard to the structure of location 94 except that the candling at location 95 is for the eggs in rows R-3 and R-4 and the candling at location 96 is for the eggs in rows R-5 and R-6.

The structural set-up for candling location 97 is the same as that for locations 94, 95 and 96 except location 97 processes six eggs as noted above and those require only six egg positioning tubes 72, assemblies 68, six light sources, and two cameras 92.

As is discussed below, the invention contemplates automatic control of the process. The process itself, however can be described briefly without reference to the automatic operation. Referring particularly to FIGS. 1 and 2, an operator can be at the operator station 12 with two trays of eggs, each tray having forty-two (or some other number as may be desired) eggs for candling. With the system in operation, a tray is inserted into the interior space 26 of the automatic candling apparatus 10 and advanced to first candling location 94. In this regard, the eggs may be advanced through the interior 26 using a conveyor 100, the operation of which may be automatically controlled by controller 112 or manually.

When a tray 20 reaches the first candling position 94, FIG. 2, it is stopped and each egg in Rows R-1 and R-2 is lifted by the corresponding telescoping tube 72 of the corresponding egg positioning assembly 68. Previously, simultaneously with the lifting of the eggs or subsequently to the lifting the cap-shaped element 78 of light source 72, FIG. 4, is displaced. The eggs abut the element 78 at the end of the lifting of the eggs and the lowering of the element 78 under control of the controller 112. With the eggs so positioned, the four cameras 92 image that half of each egg on which they are focused. The eggs are then lowered, rotated 180° and then lifted to present the opposite egg surfaces to the cameras and the imaging is repeated. The twelve now-candled eggs are lowered back onto the tray 20 and the tray 20 is advanced to the second candling location 95 and the steps are repeated, but this time the eggs of rows R-3 and R-4 are candled. The process is repeated until all the eggs have been candled and a tray 20 in which all eggs have been candled advances to the reject station 52. Here, after the images of the eggs have been analyzed by the system CPU in controller 112 as described below, those of eggs 22 which are not suitable for the purpose intended are removed from the tray 20 at position 52 by suction head 50, FIG. 1, and transferred to the second reject position 54 by a shuttle 51 where they are discarded.

The structure and its operation, using the described forty-two egg trays, are able to candle accurately over thirty thousand eggs per hour with an accuracy greatly in excess of that achieved by manual operation. As mentioned above, the candling apparatus of the invention preferably includes automatic operation.

More specifically, in a conventional candling process, a human inspector will either 1) hold the egg up in front of a bright light source or 2) place a bright light source in contact with the egg in order to see through the translucent egg shell into the egg interior. The human inspector will look for the following features to distinguish between an embryo that is suitable for use, e.g. viable and one that is infertile or inviable:

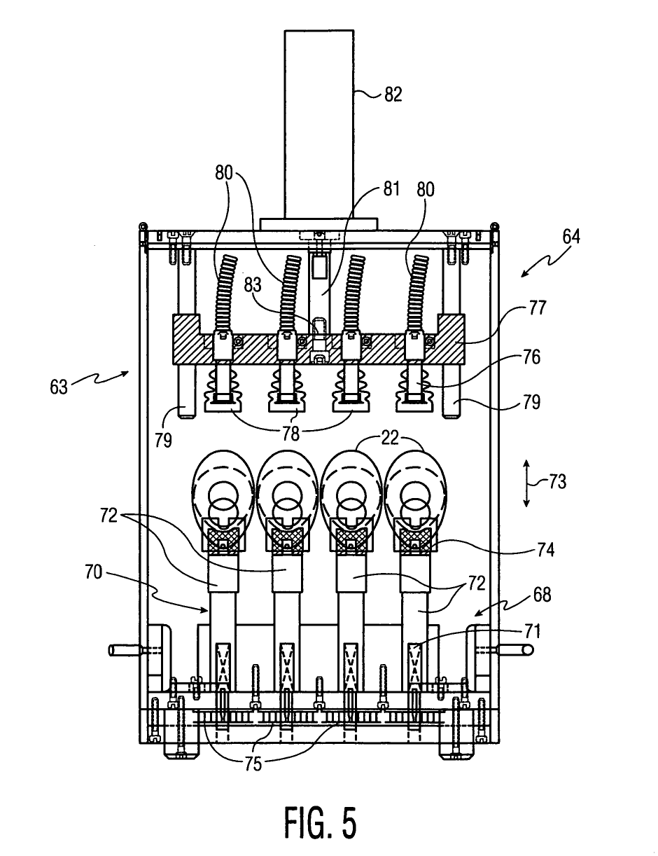

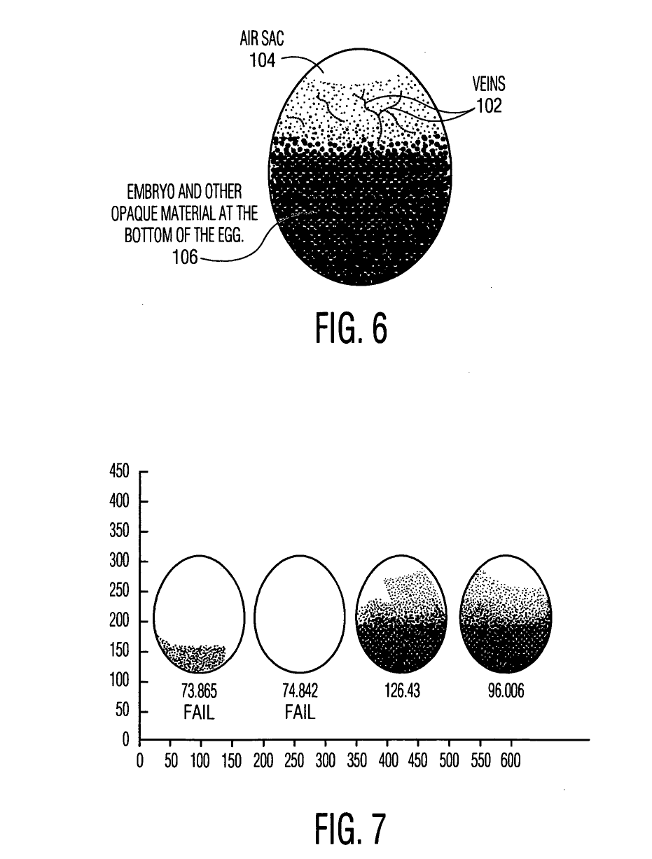

veins—FIGS. 6 and 7 (right two images, FIG. 7). A viable embryo develops veins 102 which lead from the air sac 104 at the top of the egg 22 to the embryo 106 itself at the bottom of the egg. As the embryo develops, the number and size of veins increases.

air sac—FIGS. 6 and 7 (right two images FIG. 7). A viable embryo 106 has an air sac 104 located at the top of the egg. The air sac should be well-defined with a clearly-defined edge. The air sac deteriorates after the embryo dies and is not found at all in an egg which is infertile.

opaque material at the bottom of the egg (including the embryo)—FIG. 6. As the embryo grows, the material at the bottom of the egg becomes more opaque. An infertile egg does not contain an embryo and is thus translucent at the bottom, FIG. 7, left two images.

The automated candling system of the present invention identifies the same features as a human candler would in determining the quality and viability of the embryo.

The computer system, controller 112, is connected to a video camera system including cameras 92. The computer views the live images as seen by the camera. The live images are captured into the computer and processed. Image processing is generally well known.

The image of an egg can be viewed in gray scale, in color or as the separate color components of red, green and blue. When viewed as separate color components, information about different characteristics of the egg can be more easily determined. The computer programming of the present invention employs software that uses the color components in the following manner:

Green—the green portion of the image identifies the veins. The veins on the egg are not visible in either the red or blue color component. Thus, those color components are ignored and only green is used for identifying veins

Red—the red portion of the image is used to determine which egg is being viewed. The entire area of each egg is visible in the red component.

Blue—the blue color component is used to locate cracks and the air sac. Blue light is blocked from passing through the egg except for the portion which is in contact with the air sac and locations which contain cracks.

The user has an imaging screen on which he can setup the machine. In this screen, the user sets the target age of the egg. The user will also have a check list of defects to detect. This list will include (among other things) cracks, misaligned air sacs and contamination. The user will also set a quality value threshold. Eggs rated above the quality threshold will be passed by the software. Eggs with a quality rating below the threshold will be rejected by the software. The candling machine is informed by the software of any rejected eggs by the egg's coordinates in the array on a tray so that the eggs may be removed at the end of the candling process.

The software operates as follows:

1. Capture the live image of multiple eggs from a camera.

2. Separate each image into its red, green and blue components.

3. Apply a contrast enhancement algorithm to the green component to enhance the view of vein structure.

4. Use the red component to identify the separate eggs in the image

5. For the first egg in the image, use the blue component to determine if cracks are present and if the air sac is in the top section of the egg. Optionally indicate as rejected, any egg with cracks or a misaligned air sac.

6. For the second egg in the image, use the blue component to determine if cracks are present and if the air sac is in the top section of the egg. Optionally indicate as rejected, any egg with cracks or a misaligned air sac.

7. For the third egg in the image, use the blue component to determine if cracks are present and if the air sac is in the top section of the egg. Optionally indicate as rejected, any egg with cracks or a misaligned air sac.

8. For the area within the first egg in the image, calculate the statistical information of the image including: average brightness, variance, average deviation, skewness and kurtosis. The statistical values are combined mathematically to determine a quality value or quality rating for the egg.

9. Compare the egg quality rating with the threshold value and indicate as rejected, any egg which receives a quality value below the threshold.

10. For the area within the second egg in the image, calculate the statistical information of the image including: average brightness, variance, average deviation, skewness and kurtosis. The statistical values are combined mathematically to determine a quality value or quality rating for the egg.

11. Compare the egg's quality rating with the threshold value and indicate as rejected, any egg which receives a quality value below the threshold.

12. For the area within the third egg in the image, calculate the statistical information of the image including: average brightness, variance, average deviation, skewness and kurtosis. The statistical values are combined mathematically to determine a quality value or quality rating for the egg.

13. Rotate the eggs 180° or some predetermined amount so that a different portion of the egg is visible to the cameras and repeat steps 1-12.

14. Mathematically combine the quality ratings of the multiple views of each egg. This results in a single quality rating for each egg.

15. Compare the eggs quality rating with the threshold value and indicate as rejected, any egg which receives a quality value below the threshold.

16. Send a message to the machine controller 112 system listing all rejected eggs currently within in the machine. The control system will use this information to extract the rejected eggs from their trays based on their x-y coordinates in the tray.

This software allows each user to develop an inspection process which best fits his requirements. For users with strict requirements, a higher quality threshold can be selected. Users with looser requirements may instead choose a low quality threshold to accept a larger number of eggs.

FIG. 7 is an example of four eggs which have been examined using this software. The eggs can be characterized as (from left to right): early dead, infertile, viable, viable. A quality threshold of 80 is used in this example. The software algorithm gives the two viable embryos a good score while the infertile and early dead eggs receive a low score and fail.

In FIG. 8, an example of a system for inspection of eggs for viability includes a controller 112 including a CPU 114, RAM 116 and ROM 118 which include instructions for carrying out the above program and for storing the images and related information corresponding to viable egg parameters. The egg displacement apparatus 120 includes apparatus such as conveyor 100, the lift and rotate assemblies 68, the tray shuttle 51 for displacing the tray with the identified unacceptable eggs to the reject station second position 54 from the first position 52 and for operating the suction head 50, FIGS. 1 and 2. When the eggs are in the lift position in each location 94-97, the eggs are lifted by apparatus 120, such as assemblies 68, and imaged by the imaging system which includes the cameras 92 and lights 76 and associated assemblies under control of controller 112. The images are transmitted to the controller 112 storage units and compared to the stored criteria in controller 112. The controller 112 then evaluates the eggs and determines which are good and which are bad, inviable, and fail the test. The eggs that fail are identified by controller 112 as to their coordinates in their respective tray(s).

The tray of evaluated candled eggs is then passed on to the accept-reject apparatus 124. When that tray reaches the accept-reject apparatus 124, which may comprise the reject station 16, the coordinates are fed to the suction head 50 for example at station 16, which head then is operated by controller 112 to lift the defective eggs from the tray 20. The tray 20 with the remaining good passed eggs is then passed on to a station for packaging or other disposal according to a given implementation.

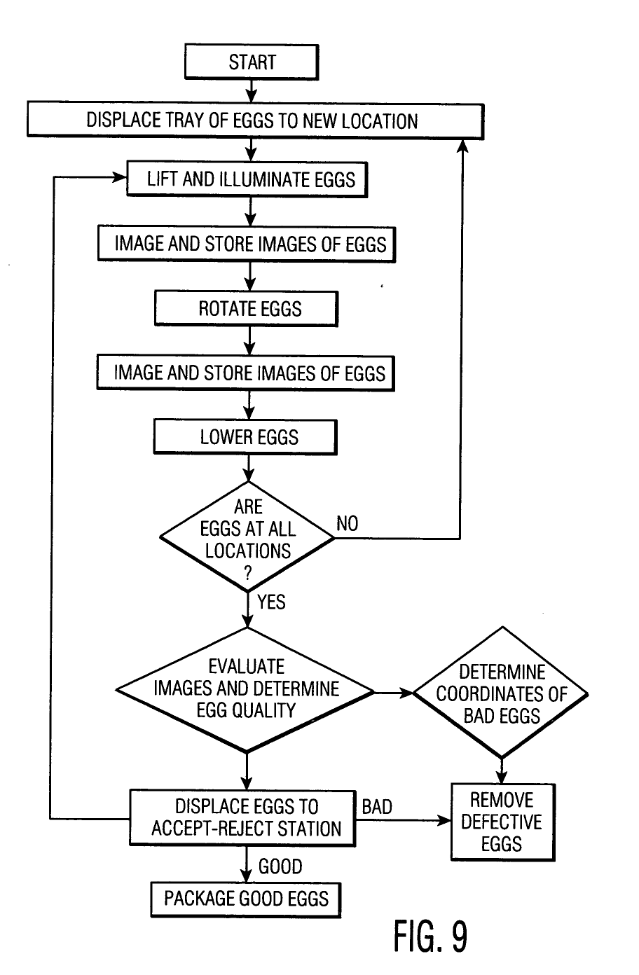

FIG. 9 shows the steps performed in the above disclosed egg candling process. All of these steps are under control of a central CPU in controller 112.

It should be understood that modifications may be made to the disclosed embodiments by one of ordinary skill. These embodiments are given by way of illustration and not limitation. It is intended that the appended claims define the invention.

What is claimed is:

1. An egg candling apparatus comprising:
an automatic imaging system for producing an image of each egg of a plurality of eggs manifesting the quality of each egg; and
an egg quality evaluation system coupled to the imaging system for automatically determining which of the imaged plurality of eggs meet a given quality value.

2. The apparatus of claim 1 including an egg separating system coupled to the imaging system for separating those eggs that have at least the given quality value from those that do not have at least that value.

3. The apparatus of claim 1 wherein the imaging system includes a plurality of digital video cameras focused on the eggs.

4. The apparatus of claim 1 wherein the imaging system includes a plurality of egg positioning assemblies for lifting and rotating each egg, a plurality of egg illuminating assemblies to illuminate each lifted egg and a plurality of digital video cameras to image the illuminated egg interiors.

5. The apparatus of claim 4 wherein each egg is first imaged in a first relative angular orientation to at least one of the cameras and then rotated to a second angular orientation for imaging by at least one of the cameras.

6. The apparatus of claim 4 including an egg holding tray for holding the eggs in rows and columns, each tray having an opening therein for receiving therethrough a corresponding egg lifting and rotating assembly.

7. The apparatus of claim 4 wherein the illuminating assemblies include a light assembly for illuminating the eggs, the egg positioning assemblies for abutting each egg against a corresponding light assembly, and including a drive motor for selectively rotating and lifting the eggs for imaging by the cameras.

8. The apparatus of claim 7 wherein the eggs are positioned in a plurality of columns and rows on a tray, the positioning assemblies, lights and cameras being arranged to selectively image the eggs in at least one row of eggs on a tray.

9. The apparatus of claim 8 including a plurality of locations wherein all of the eggs on the tray are imaged in successive ones of the locations, each location for imaging at least one different row of eggs of the plurality of rows.

10. The apparatus of claim 9 wherein each camera is arranged to image a plurality of eggs in a row.

11. The apparatus of claim 1 wherein the imaging system is arranged to image at least one of the veins, air sac and location of the embryo of each egg and including a controller to compare the veins, air sac and embryo to a reference.

12. The apparatus of claim 1 including:
a conveyor;
at least one tray holding a plurality of eggs in rows and columns for displacement by the conveyer;
at least one candling station for canding the plurality of eggs, the at least one station including the imaging system, the imaging system including a digital video camera for imaging at least a portion of the plurality of eggs at a time, the imaging system further including egg lifting and rotating assemblies for lifting the portion of the eggs and an illuminating system for illuminating the interiors of the lifted portion of eggs for the camera imaging; and
a controller for operating the imaging and illuminating systems and the quality evaluation system.

13. The apparatus of claim 1 including a controller for operating the imaging and evaluation systems.

14. The apparatus of claim 13 wherein the controller includes a CPU and a data base containing reference data identifying the features of a viable egg and programmed instructions for comparing the features of each of the illuminated eggs with reference data features.

15. The apparatus of claim 14 wherein the features in the reference data include veins, air sac location in the egg and embryo.

16. The apparatus of claim 14 wherein the evaluation system is arranged to assign values to each the features and for assigning a threshhold value of the assigned values for an acceptable egg.

17. The apparatus of claim 1 including an egg separation system for separating eggs which meet the given quality value from those that do not meet the quality value.

18. The apparatus of claim 1 wherein the imaging system includes a computer having a plurality of different programmed settable reference quality values each defining an aggregate value of a plurality of acceptable egg features and at least one digital video camera for imaging the egg features of the plurality of eggs for evaluation by the computer, the computer being responsive to the at least one camera images applied thereto for assigning each of the plurality of eggs an aggregate quality value, the computer for comparing the assigned aggregate quality value to the assigned reference value to provide an output value manifesting the compared values for each egg.

19. The apparatus of claim 18 wherein the aggregate assigned values are the combined values of egg features including veins, air sac location in the egg and the presence of an embryo.

20. The apparatus of claim 17 including a tray for carrying the plurality of eggs, the eggs on the tray each having a given different coordinate defining its location on the tray, the imaging system for identifying the coordinates of bad eggs that do not meet the given quality value, the separation system for removing the bad eggs from the tray based on their coordinates in the tray.

21. The apparatus of claim 1 wherein the imaging system includes a plurality of trays each carrying the first plurality of eggs, the plurality of trays in combination for carrying a second plurality of eggs, the imaging system for imaging a further plurality of eggs on each of the trays, the eggs of the further plurality being different on each tray such that the further plurality of eggs imaged on each tray together result in imaging all of the first plurality of eggs on a tray.

22. The apparatus of claim 21 wherein the eggs on each tray are assigned rows and columns, a first imaging subsystem for imaging a first row of eggs on a first tray forming a first further plurality of eggs, a second imaging subsystem for imaging a second row of eggs on the first tray forming a second further plurality of eggs, and further imaging subsystems for imaging at least one further row on the first tray of still additional further plurality of eggs so that all the subsystems together image all of the eggs on the first tray.

23. An egg candling apparatus comprising:
a tray for holding a plurality of eggs in rows and columns having defined coordinates for each egg;
a conveyor for conveying the tray;
a digital imaging device for creating images of the conveyed interior of different ones of the eggs;
a data base of reference features of an acceptable egg;
a controller responsive to the images of each egg arranged to compare the egg features of the supplied images to the reference features in the data base to produce a quality value representing the quality of each egg and for determining whether an egg is acceptable;
the controller for identifying the coordinates of each unacceptable egg; and
a disposable system responsive to the controller for discarding the unacceptable eggs.

24. The apparatus of claim 23 including an egg lifting and rotating assembly for lifting and rotating each egg on the tray for said imaging.

25. The apparatus of claim 24 including an illuminating system associated with each lifting and rotating assembly, each lifting and rotating assembly being responsive to the controller to lift an egg off of the tray toward the associated illuminating system to illuminate the lifted egg interior in a first angular position relative to the imaging device, each lifting and rotating assembly for rotating each egg to a second angular position relative to the imaging device in response to the controller for imaging a lifted egg in the second angular position, the controller for creating an aggregate quality value for each egg based on the images in the first and second angular positions.

26. The apparatus of claim 23 wherein each egg includes veins, an air sac and an embryo and wherein the features include the quality of the veins, the location of the air sac and the presence and location of the embryo.

27. A method for candling eggs comprising automatically producing an image manifesting the quality of each egg of a plurality of eggs and automatically evaluating the image of each egg to determine which eggs have at least a given quality value.

28. The method of claim 27 including separating the eggs that do not have the given quality value from those that have at least that value.

29. The method of claim 27 wherein the imaging step includes imaging each egg with a digital camera.

29. The method of claim 27 wherein the imaging step includes lifting each egg, illuminating each lifted egg, imaging the illuminated egg at a first angular position, rotating the imaged egg to a second angular position and imaging the egg at the second angular position.

30. The method of claim 28 including imaging a plurality of eggs in a row.

31. The method of claim 30 including placing the eggs in a plurality of rows defining a plurality of columns, and wherein the imaging includes imaging the eggs in different rows at different times.

32. The method of claim 31 including imaging the eggs in different rows with different cameras.

33. The method of claim 31 including imaging different eggs in a row with a different camera.

34. The method of claim 33 including simultaneously imaging eggs in a plurality of rows.

35. The method of claim 34 including sequentially imaging eggs in different rows.

36. The method of claim 35 including imaging eggs in a first row or rows with a first camera, imaging eggs in a second row or rows with a second camera and imaging eggs in a third row or rows with a third camera.

37. The method of claim 36 including imaging all said rows simultaneously.

38. The method of claim 37 wherein the eggs being imaged in the first row are in different columns from the eggs in the second row which are in different columns from the eggs in the third row.

39. The method of claim 33 including imaging the eggs of a row simultaneously.
